(19) United States
(12) Patent Application Publication
(10) Pub. No.: US 2016/0249832 A1
(43) Pub. Date: Sep. 1, 2016

(54) Activity Classification Based on Classification of Repetition Regions

(71) Applicant: Amiigo, Inc., Mountain View, CA (US)

(72) Inventors: Abraham Luke Carter, Palo Alto, CA (US); David Jennings Scott, Palo Alto, CA (US); Ehsan Azarnasab, Salt Lake City, UT (US)

(21) Appl. No.: 14/634,685

(22) Filed: Feb. 27, 2015

(51) Int. Cl. A61B 5/11; A61B 5/00

(52) U.S. Cl. CPC A61B 5/1118; A61B 5/1123; A61B 5/4519; A61B 5/7264; A61B 5/0022; A61B 5/6801; A61B 5/7246

(57) ABSTRACT

A wearable device allows the tracking of human movements during activity, such as while exercising or playing a sport. To improve user experience with the wearable device, an activity classification server classifies activities and repetitions of the activity. To further improve user experience, the activity classification server can prompt the user to perform an activity and identify the activity while the user performs the prompted activity. The user can also indicate to the device an election to perform a particular activity without being prompted by the device and the user can classify the activity by indicating to the device what activity the user will perform.

### BACKGROUND

This invention relates generally to wearable devices and more specifically to classification of activities performed while using one or more wearable devices.

Many devices have been developed for tracking various physiological parameters and activity information during a user's workout or other user activities. For example, heart rate monitors can detect a user's heart rate, pulse oximeters can detect the oxygen saturation of a user's hemoglobin, blood glucose monitors can detect the glucose level in a user's blood, etc., and the various physiological and activity information can be used to classify an activity performed by the user.

To track these various parameters, the user is required to wear or carry a specialized device with a potentially uncomfortable or inconvenient form factor that can both receive raw data from the sensors and process the raw data to generate useful information displayable to the user. However, there is a limited amount of information that can be collected by such a specialized device worn by the user if the various activities performed by the user while using the specialized device are unknown.

### SUMMARY

Users can wear one or more activity-tracking devices while performing an activity and have the activity classified by an activity classification server. An activity classification server receives raw data from the activity-tracking devices where the raw data includes data from each of the activity-tracking devices and represents activity (e.g., motion) data of a user using the activity-tracking devices through points (e.g., points in a stream of data from the activity-tracking device or in a graphical representation of the data, such as peaks or valleys). Each point in the raw data is associated with a time stamp and amplitude. The received raw data can be data collected when the user is using the activity-tracking devices while performing an activity (herein referred to as “activity regions”), data collected when the user is resting (e.g., “idle regions”) and performing an activity or any combination thereof.

A set of feature points is identified in the raw data or the identified activity region in the raw data. A feature point is a point in the raw data identified based on the amplitude of the point, the time stamp, associations between the point and other points in the raw data, or any combination thereof.

A repetition (“rep”) region in the raw data is determined based on the set of feature points. A rep region is a region in the raw data that includes points within an interval of time within each other. As an example, the rep region represents a repetition or ‘reps’ done by the user during an exercise (e.g., a set of ten bicep curls, a single bicep curl, a number of pirouettes performed by a dancer, a single pirouette performed by a dancer, etc.), and can be determined based on whether a threshold number of associations have been made between various feature points within the set of feature points in the rep region with other feature points in another rep region.

The activity classification server classifies the rep region as a repetition type and can classify the rep region as a repetition type based on the threshold number of associations made between various feature points in the rep region. A repetition type can be any movement in an activity such as a sport or an exercise. For example, repetition types in basketball can include dribbling, running, shooting, passing, stepping, and standing. Herein, a rep region that is classified as a repetition type of “dribble” can be referred to as a “dribble” rep region.

Based on the classified rep region as the repetition type, the activity region is associated with an activity type. Following the example from before, when a user uses the plurality of activity-tracking devices while playing basketball, there may be a plurality of rep regions in the activity region of the raw data such as a “dribble” rep region, five “step” rep regions, and a “shoot” rep region and, based on the combination of those types of repetitions, the activity region is associated with an activity type such as “basketball.” The activity classification server may also determine that an association of the activity region with an activity type cannot be made based on the classified repetition type. Thus, in response to this determination, the activity classification server can determine an additional rep region in the raw data based on the set of feature points, classify the additional rep region as a repetition type, and associate the activity region with an activity type based on the two classifications of the rep region and the additional rep region.

### BRIEF DESCRIPTION OF THE DRAWINGS

FIG. 1 is a block diagram of a system environment in which one or more devices used for activity-tracking and an activity classification server operate, in accordance with an embodiment.

FIG. 2 is a block diagram of an architecture of the activity classification server, in accordance with an embodiment.

FIG. 3 is a block diagram of the plurality of activity-tracking devices illustrated in FIG. 1, in accordance with an embodiment.

FIG. 4 is a method of classifying an activity region as an activity type, in accordance with an embodiment.

FIG. 5 is an example of raw data including an activity region, an idle region, and a rep region and feature points in the raw data, in accordance with an embodiment.

FIG. 6 is an example of feature points in raw data indicating a repetition is associated with muscle fatigue or higher resistance, in accordance with an embodiment.

The figures depict various embodiments of the present invention for purposes of illustration only. One skilled in the art will readily recognize from the following discussion that alternative embodiments of the structures and methods illustrated herein may be employed without departing from the principles of the invention described herein.

### DETAILED DESCRIPTION

### System Architecture

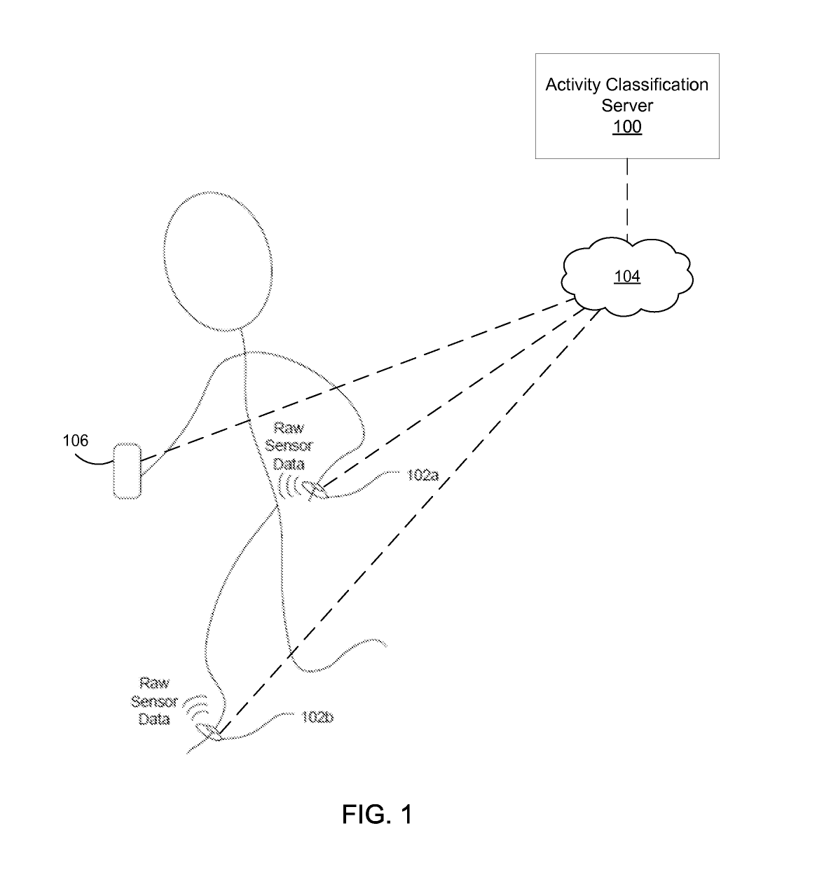

FIG. 1 is an example block diagram of a system environment in which one or more devices used for activity-tracking (“activity-tracking devices”) and an activity classification server 100 operate. The system environment shown by FIG. 1 includes an activity classification server 100, a plurality of wearable or activity-tracking devices 102A and 102B, and a network 104. In alternative configurations, different and/or additional components may be included in the system environment. For example, there may only be a single wearable or activity-tracking device or more than two, and there may also be a portable computing device 106.

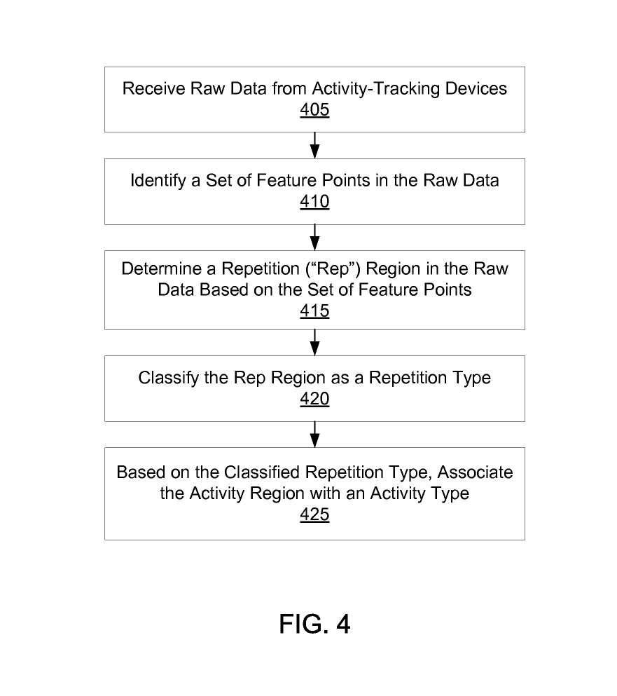

The activity classification server 100 receives raw data from one or more activity tracking devices 102 and classifies activities and repetitions in activities using the raw data. The classification and other suitable information based on the classification can be presented to users of the activity classification server 100 via one or more portable computing devices 106. As further described below in conjunction with FIG. 4, the activity classification server 100 receives raw data, identifies activity regions in the raw data, identifies repetition regions in the activity regions, and classifies the activity regions, repetition regions, or both. In one embodiment, the activity classification server 100 is a cloud-based computing system with one or more servers that performs most or all of the data processing and analysis of raw data collected by the activity tracking devices 102. This provides a benefit in that the devices 102 can collect much more data than they might otherwise be able to if much or most of the processing were performed on those devices 102 themselves. For example, it is possible to collect resting heart rate data as opposed to collecting only heart rate data during a particular activity of interest because the devices 102 do not need to store or perform the analysis on this data, and this data is instead sent to the server 100 for storage/analysis. This allows for a more accurate picture of a user's activities and allows for tracking of a wider variety of activity types.

The portable computing device 106 can be a computing device operated by the user (e.g., Smartphone, laptop, tablet, etc.) or an application executed on a client device and capable of transmitting and/or receiving data via the network 104. Thus, the portable computing device 106 is configured to communicate via the network 104. In some embodiments, a portable computing device 106 is not included as a part of the system environment. The activity-tracking devices 102A and 102B each can be worn and used by a user during a workout or an activity, and each can include a housing and one or more sensors attached to the housing.

As shown in FIG. 1, activity-tracking device 102A is worn by a user on the user's wrist (e.g., as a bracelet) and wearable device 102B is worn by the user on the user's shoe (e.g., as a shoe clip). Thus, activity-tracking device 102A captures activity information, physiological information, or both activity and physiological information of the user's arm and activity-tracking device 102B captures activity information, physiological information, or both activity and physiological information of the user's leg. Having an activity-tracking device on the upper half or portion of the user's body and on the lower half of the user's body allows for more accurate classification of activity data and classification of more complex activities as it is difficult to determine what activity a user is performing based just on a user's arm or leg movements alone. In alternative embodiments, activity-tracking device 102A is worn by a user somewhere on the user's upper half of the body and activity-tracking device 102B is worn by the user somewhere on the user's lower half of the body. Although FIG. 1 illustrates two activity-tracking devices 102A and 102B, alternative embodiments include one wearable device or three or more wearable devices that can be worn by a user on the user's elbow, hip, knee, head, or any other portion of the user's body. Activity tracking devices can also be placed on tools or sporting equipment used by a user during an activity (e.g., on a weight stack in a gym, on a golf club or tennis racquet, on a gardening tool, etc.).

The use of and interaction between two or more wearable devices for activity tracking is described in more detail in U.S. patent application Ser. No. 13/846,662 filed on Mar. 18, 2013 (describing, for example, correlating accelerometer data to known movements), U.S. patent application Ser. No. 14/172,726 filed on Feb. 4, 2014 (describing, for example, identifying physiological parameters from raw data received wirelessly from a sensor), U.S. patent application Ser. No. 14/184,597 filed on Feb. 19, 2014 (describing, for example, synchronizing accelerometer data received from multiple accelerometers and dynamically compensating for accelerometer orientation), U.S. patent application Ser. No. 13/891,699 filed on May 10, 2013 (describing, for example, updating firmware to customize the performance of a wearable sensor device for a particular use), U.S. patent application Ser. No. 14/275,493 filed on May 12, 2014 (describing, for example, a platform for generating sensor data), and U.S. patent application Ser. No. 14/279,140 filed on May 5, 2014 (describing, for example, correlating sensor data obtained from a wearable sensor device with data obtained from a smart phone), each of which is incorporated by reference herein in its entirety.

The activity classification server 100 and activity-tracking devices 102A and 102B are configured to communicate via the network 104, which may comprise any combination of local area and/or wide area networks, using both wired and/or wireless communication systems. In one embodiment, the network 104 uses standard communications technologies and/or protocols. For example, the network 104 includes communication links using technologies such as Ethernet, 802.11, worldwide interoperability for microwave access (WiMAX), 3G, 4G, code division multiple access (CDMA), digital subscriber line (DSL), etc. Examples of networking protocols used for communicating via the network 104 include multiprotocol label switching (MPLS), transmission control protocol/Internet protocol (TCP/IP), hypertext transport protocol (HTTP), simple mail transfer protocol (SMTP), and file transfer protocol (FTP). Data exchanged over the network 104 may be represented using any suitable format, such as hypertext markup language (HTML) or extensible markup language (XML). In some embodiments, all or some of the communication links of the network 104 may be encrypted using any suitable technique or techniques. In some embodiments, one or more of the devices 102A and 102B are configured to communicate directly with each other or directly with the portable computing device 106 via, for example, a BLUETOOTH® connection.

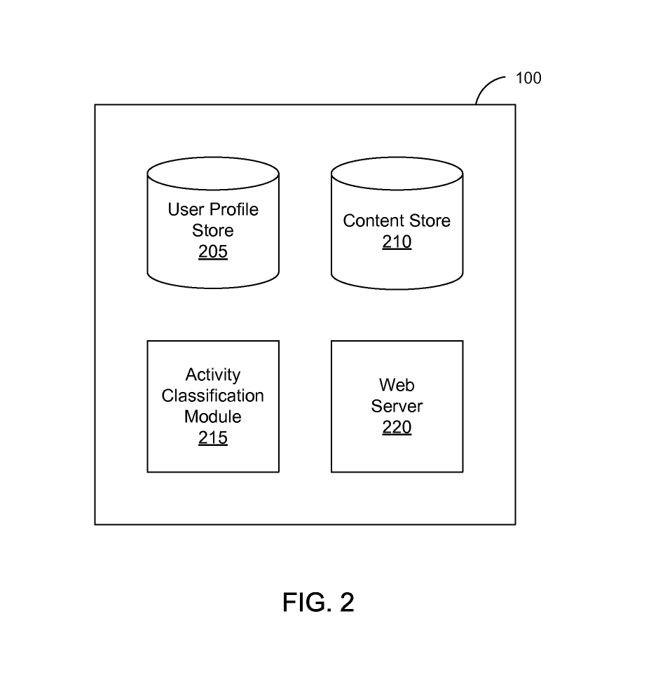

FIG. 2 is a block diagram of an architecture of the activity classification server 100. The activity classification server 100 shown in FIG. 2 includes a user profile store 205, a content store 210, an activity classification module 215, and a web server 220. In other embodiments, the activity classification server 100 may include additional, fewer, or different components for various applications. Conventional components such as network interfaces, security functions, load balancers, failover servers, management and network operations consoles, and the like are not shown so as to not obscure the details of the system architecture.

Each user of the activity classification server 100 is associated with a user profile, which is stored in the user profile store 205. A user profile includes information about the user such as information provided by the user and information inferred by the activity classification server 100. A user profile in the user profile store 205 may also maintain associations with activities performed by the corresponding user while using one or more activity-tracking devices 102.

The content store 210 can store data from one or more activity-tracking devices 102, such as raw data received from one or more activity-tracking devices 102, activity regions identified in raw data received from one or more activity-tracking devices 102, classifications of activity regions (as activity types), repetition regions identified in activity regions, classifications of repetition regions (as repetition types), analysis of raw data, and any combination thereof. The content store 210 can also store associations of any data stored in the content store 210 with a user profile of a user associated with the data. This content associated with a user profile of each user can be used by the server 100 to simplify classification of activities since the content associated with the profile of a user can indicate what types of activities that user normally performs (e.g., plays basketball and swims), providing a starting point for classification of a likely activity that the user is performing.

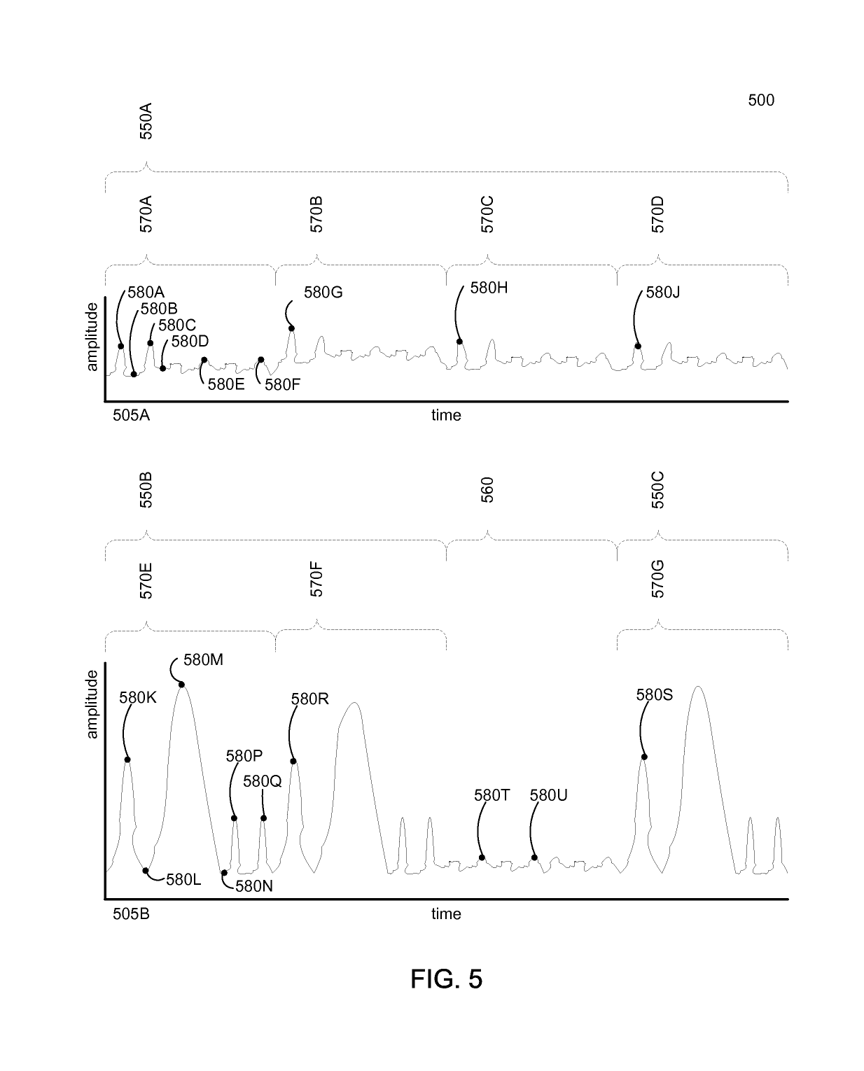

The content store 210 can also store a reference database of previously classified activity regions as activity types, repetition regions as repetition types, and any combination thereof. The reference database can be user-specific and include previously determined classifications of a specific user and, therefore, can be associated with a user profile of the specific user. Alternatively, the reference database can be a generic database including a combination of previously determined classifications of one or more users of the activity classification server 100. The reference database provides examples of activity regions against which new activity data collected can be compared to make easier the classification of activities in the new data. For example, bicep curls may have a typical, characteristic shape when graphically represented and so this reference shape can be used in the future to easily classify new data having the same shape as bicep curls. Similarly, different users may perform different activities a bit differently such that one user's bicep curl has a somewhat different shape than another's. Thus, user-specific reference databases can be used as a reference for that particular user's way of performing the activity, as further described below in conjunction with FIG. 5.

The activity classification module 215 performs analysis techniques on raw data received from one or more activity-tracking devices 102 and classifies identified activity regions and/or repetition regions in activity regions as activity types and/or repetition regions, respectively. The activity classification module 215 can use various machine learning algorithms, such as any suitable pattern recognition technique including classification algorithms, clustering algorithms, Bayesian networks, Markov random fields, Kalman filters, sequence labeling algorithms, linear discriminate analysis, support vector machines, principal component analysis, wavelet transforms, and any other suitable pattern recognition or classification systems. Thus, the activity classification module 215 identifies characteristics or feature points in the raw data, determines regions (activity and repetition), and classifies those regions, as further described below in conjunction with FIG. 4.

The web server 220 links the activity classification server 100 via the network 104 to a portable computing device 106. The web server 220 serves web pages, as well as other content, such as JAVA®, FLASH®, XML and so forth. A user may send a request to the web server 220 to access information that is stored in the content store 210. Additionally, the web server 220 may provide application programming interface (API) functionality to send data directly to native portable computing device operating systems, such as IOS®, ANDROID™, WEBOS® or BLACKBERRYOS.

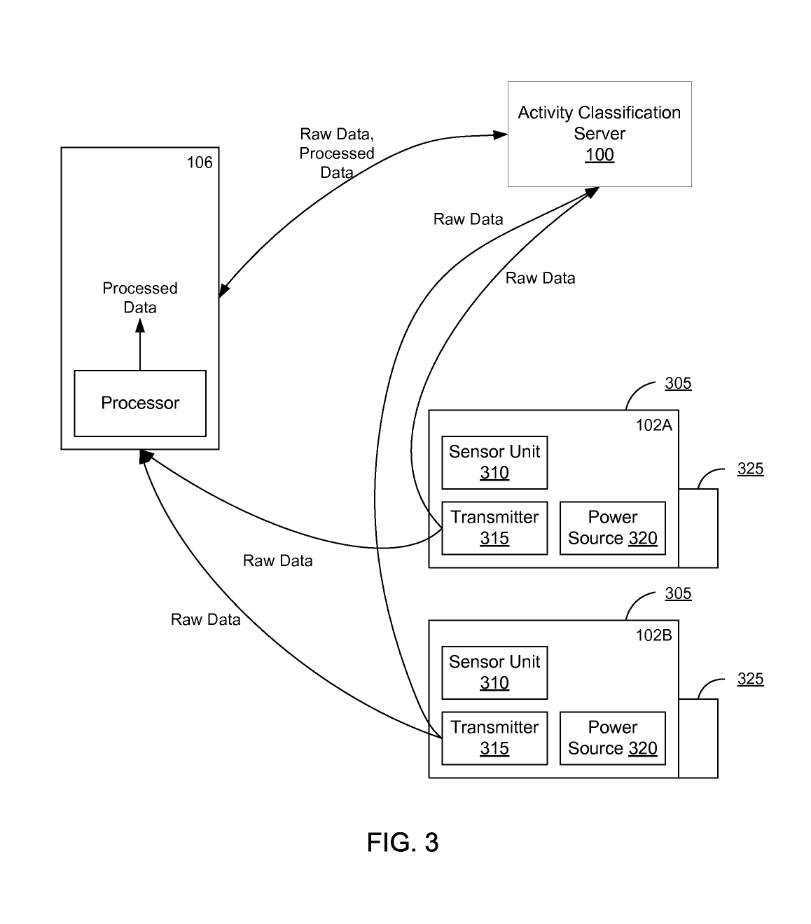

FIG. 3 is a block diagram of the plurality of activity-tracking devices 102 as illustrated in FIG. 1. Raw data captured by the plurality of activity-tracking devices 102A and 102B are wirelessly transmitted via the network 104 to the activity classification server 100, a portable device 106, or any combination thereof. The portable computing device 106 or an application executed on the portable computing device 106 processes the raw data and can present the processed data to a user of the portable computing device 106 via, for example, the application executing on the portable computing device 106.

Each of the devices in the plurality of activity-tracking devices 102A and 102B includes a housing 305 with a sensor unit 310, a transmitter 315, a power source 320, and a coupling mechanism 325. The sensor unit 310, transmitter 315, the power source 320, and coupling mechanism 325 are attached to the housing 305. In alternative embodiments, one or more of the plurality of wearable devices 102A and 102B can also include a display, one or more light-emitting diodes (LEDs), a processor, a memory, or any combination thereof. In other embodiments, each device may include additional, fewer, or different components for various applications. Conventional components such as network interfaces, security functions, and the like are not shown so as to not obscure the details of the devices.

The sensor unit 310 includes a blood glucose sensor, a pulse oximeter, a skin temperature sensor, a blood pressure sensor, a single-axis accelerometer, a multi-axis accelerometer, a global positioning system (GPS), a gyroscope, any other suitable sensor for capturing motion information associated with the user or physiological or biometric data, and any combination thereof.

The transmitter 315 transmits raw data to a portable computing device 106 or the activity classification server 100 at various frequencies based on characteristics of the raw data. Characteristics of raw data or feature points in the raw data describe changes in patterns in the raw data such as a data point in the raw data that differs from one or more previous data points in the raw data. For example, if the raw data describes acceleration in a vertical direction, a characteristic of the raw data can describe magnitude of the acceleration and, therefore, a change in pattern can be a change in magnitude. Other examples of characteristics include changes in VO2 levels, lactate levels, blood glucose levels, duration, and any other suitable characteristic of data that can be measured by the one or more sensors in the sensor unit 210. In various examples, the characteristics of raw data can be used to detect patterns to identify whether a user is performing an activity, to identify whether a user is performing an activity properly or safely, to identify whether a user is suffering a condition while performing an activity, to identify whether the user may have early onset of a disease, or to enhance patient care monitoring.

Characteristics can be detected and analyzed in the processor using instructions stored in memory in one embodiment. Embodiments of a processor include a microprocessor, a central processing unit (CPU), a graphics processing unit (GPU), a digital signal processor (DSP), one or more application specific integrated circuits (ASICs), one or more field programmable gate arrays (FPGAs), one or more programmable logic devices (PLDs), or any combination thereof. Embodiments of memory include flash memory, random-access memory (RAM), read-only memory (ROM), or any combination thereof. Alternatively, instructions can be stored in the processor's cache instead of in memory.

The transmitter 315 also transmits the raw data to the activity classification server 100 that communicates with a portable computing device 106 or an application executing on the portable computing device 106. The activity classification server 100 can store the received raw data and automatically process the raw data as further described below in conjunction with FIG. 4. Then, the processed data can be transmitted to the portable computing device 106 to be presented to the user.

In one embodiment, the power source 320 includes a lithium polymer battery or any other suitable battery. Alternatively, the power source 320 can include a charging apparatus built into the wearable device 102 in addition to the battery. The coupling mechanism 325 can be a ring-like structure (e.g., a wristband, armband, legband, headband, neck strap, etc.), a clip, Velcro, or any other suitable customizable and/or attachable mechanism.

### Classifying an Activity Region as an Activity Type Based on Rep Regions

FIG. 4 is a method of classifying an activity region as an activity type. In various embodiments, the steps described in conjunction with FIG. 4 may be performed in different orders. Additionally, in some embodiments, the method may include different and/or additional steps from those shown in FIG. 4. The functionality described in conjunction with the activity classification server 100 in FIG. 4 may be provided by the activity classification module 215, in one embodiment, or may be provided by any other suitable component, or components, in other embodiments.

The activity classification server 100 receives 405 raw data from one or more activity-tracking devices 102. The raw data includes data from each of the one or more activity-tracking devices 102 and represents activity (e.g., motion) data of a user using the one or more activity-tracking devices 102 through points in the data (e.g., points in a graphical representation of the data). The point can be a measurement taken by a sensor in an activity-tracking device 102. For example, if the sensor is a motion sensor such as an accelerometer, the point can be a measurement of change in a unit in a specified axis at a point in time such as a change in ten units in the x-axis direction after ten seconds of using the activity-tracking device 102 including the motion sensor. If the sensor is a temperature sensor such as a thermocouple, the point can be a measurement of the temperature at a specified point in time such as 95° F. after fifteen seconds using the activity-tracking device 102 including the temperature sensor. Each point in the raw data is associated with a time stamp and amplitude (e.g., measure of change in unit such as inches in a direction, temperature measurement). Thus, for motion data, the amplitude represents how much the activity-tracking device 102 is moving and represents motion of a user using the activity-tracking device 102. In one embodiment, for a plurality of activity-tracking devices 102, the raw data includes a plurality of data streams associated with each of the plurality of activity-tracking devices 102. Each data stream in the plurality of data streams can be associated with one of the plurality of activity-tracking devices 102. For example, in one embodiment, points in a data stream associated with a first activity-tracking device 102A can be stored with an association to an identifier identifying the first activity-tracking device 102A and points in a second data stream associated with a second activity-tracking device 102B can be stored with an association to a second identifier identifying the second activity-tracking device 102B. In an alternative embodiment, the raw data is a combined data stream of each of the one or more activity-tracking devices 102. Similarly, each point in the combined data stream can be associated with an identifier identifying the activity-tracking device 102 from which the point was acquired.

In one embodiment, the received raw data is only data from when the user using the activity-tracking devices 102 and, thus, includes an activity region. Alternatively, the received raw data includes one or more activity regions and one or more idle regions. An activity region in a data stream of the raw data can be associated with a threshold number of points with amplitudes that each exceed a threshold amplitude within an interval of time and an idle region includes a second threshold number of points associated with amplitudes less than a second threshold amplitude. For example, if the received raw data includes one hundred points associated with amplitudes above X and a following set of forty points associated with amplitudes less than X, amplitudes that are less than a threshold percentage of X (e.g., 10% of X) or any combination thereof, then the forty points can be identified as an idle region and the first one hundred points can be identified as an activity region. Thus, if the user is resting, the raw data tracked by the activity-tracking devices 102 would include points that represent an idle region. Alternatively, an activity region in a data stream of the raw data can be identified as an activity region if a second data stream of the raw data at an interval of time aligning with the activity region is associated with a threshold number of points with amplitudes that each exceed a threshold amplitude within the interval of time and an idle region includes a second threshold number of points associated with amplitudes less than a second threshold amplitude.

An activity region in the received raw data may also be identified based on a standard deviation of the received raw data over time. In other embodiments, the activity region can be identified based on variance or any other suitable measure of difference of amplitudes of points in the received raw data. Using standard deviations as an example, if a first interval of time in the received raw data includes points with a greater standard deviation than points in a second interval of time in the received raw data, the points in the first interval of time (or first region) and the points in the second interval of time (or second region) can be associated with either activity regions or idle regions based on the difference between the standard deviations. If the first region has a standard deviation greater than a standard deviation of the second region by at least a threshold deviation, then the first region can be identified as an activity region. The standard deviation of the second region can be compared to a second threshold deviation to determine whether the second region is an activity region or an idle region. In one embodiment, the second threshold deviation can be close to zero and, therefore, if the standard deviation of the second region is less than the second threshold deviation, then that indicates that there is minimal movement and the second region can be classified as an activity region. Otherwise, the second region can be identified as an activity region as well.

When an activity region is identified, the activity classification server 100 can perform a preliminary analysis to identify repetitive regions in the raw data, to identify repetitive regions in the activity region, to identify repetitive regions in the raw data before the activity region, to identify repetitive regions in the raw data after the activity region, or any combination thereof. For example, if a user is exercising while using the activity-tracking devices 102 and the exercise includes repeating three different motions such as squats, lunges, and sit-ups at least twice, then the activity classification server 100 identifies that an activity region includes two repetitive regions (e.g., two regions corresponding to a motion that is the same, which may turn out to be the squat motion once the classification is complete) and continues to analyze and classify only one of the two repetitive regions, thus reducing processing and computation time. The activity classification server 100 can then analyze the regions before the activity region and, if the regions include an additional activity region, identify whether the additional activity region includes similar repetitive regions as previously classified, thus reducing processing and computation time as well. This saves processing and computing time because the server 100 does not have to expend the resources to separately identify that a particular repetitive motion corresponds to a squat motion across every repetition of the squat motion, but simply classifies one of the repetitive regions to be the squat motion. The other repetitive regions can be determined to be the squat motion simply because they have a pattern that generally matches the first region that was classified as the squat motion. Each repetitive region is associated with a threshold interval of time and a threshold number of points. Thus, if there are two repetitive regions, each repetitive region exceeds at least the threshold interval of time in duration and includes at least the threshold number of points.

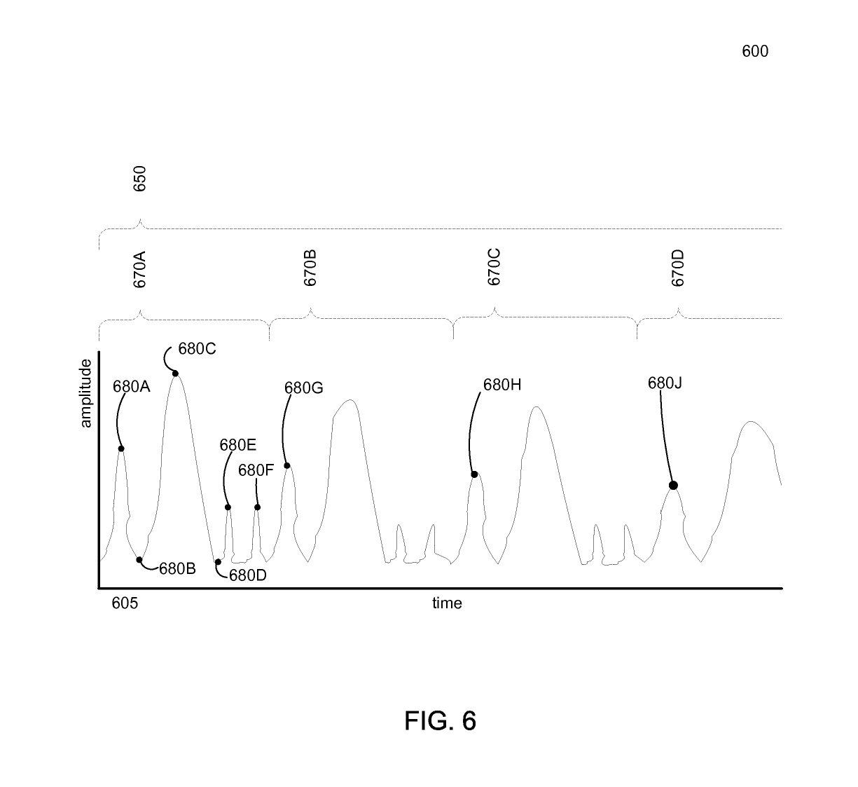

The activity classification server 100 can also perform analysis to identify differences in the two repetitive regions (e.g., differences between the first repetitive region that was classified to be a squat motion and the second repetitive region of squats). For example, if the raw data in the first repetitive region of the activity region is cleaner or smoother than the second repetitive region of the activity region, the activity classification server 100 can store information indicating the second repetitive region is associated with muscle fatigue, higher resistance, or any combination thereof. As another example, a user may be exercising while using the activity-tracking devices 102 and the exercise may include three repetitions of bicep curls. The first repetition may be cleaner or smoother than the second repetition and the third repetition. Alternatively, the first repetition may include points with higher amplitudes than points in the second and third repetition. In either of these cases, the activity classification server 100 can store information indicating the second and third repetitions are associated with muscle fatigue, higher resistance, or any combination thereof. An example of information indicating a repetition is associated with muscle fatigue or higher resistance is further described below in conjunction with FIG. 6. The activity classification server 100 can then use the information to determine how many calories were burned, provide suitable training recommendations, provide notifications to the user regarding the muscle fatigue or higher resistance, or provide any combination thereof.

A set of feature points are identified 410 in the raw data or the identified activity region in the raw data. A feature point is a point in the raw data and, thus, also includes a time stamp and amplitude. A feature point in the raw data can be a point associated with an amplitude that exceeds a threshold amplitude, with an amplitude that does not exceed a threshold amplitude, or with any other suitable amplitude. In one embodiment, the threshold amplitude to which a feature point is compared (or first threshold amplitude) is different from the threshold amplitude to which a point in an activity region is compared (or second threshold amplitude) as described previously when an activity region is identified. For example, comparing the points in the raw data to the first threshold amplitude differentiates activity regions from idle regions for idle regions that are most likely made up of points with similar and low amplitudes (e.g., flat line) in the raw data. Then, comparing the points in an activity region in the raw data to the second threshold amplitude can be used to differentiate rep regions. For example, comparing the points to the second threshold amplitude can identify peaks in the raw data and those points in those peaks can be identified as feature points. In other embodiments, the threshold amplitude to which a feature point is compared can be the same as the threshold amplitude to which a point in an activity region is compared as described previously when an activity region is identified.

A feature point associated with a time stamp can also be identified based on a second feature point in the raw data associated with a second time stamp that is an interval from the time stamp. In one embodiment, associations between feature points within the set of feature points can be determined based on similarity in amplitudes, the time stamps of the feature points and whether the time stamps are at least a predetermined interval from each other. As an example of an association between points, if there is a first point with an amplitude of X that is within a first interval of time from a second point with an amplitude of Y and a third point with an amplitude of X+ε that is within a second interval of time from a fourth point with an amplitude of Y+δ where the first interval of time and the second interval of time differ in seconds or microseconds and ε and δ are less than a threshold percentage (e.g., 10%, 1%, 0.1%, or any other suitable percentage) from X and Y, respectively, then the first point is determined to be similar to the third point and the second point is determined to be similar to the fourth point.

A repetition or rep region in the raw data is determined 415 based on the set of feature points. A rep region is a region in the raw data that includes points within an interval of time within each other. As an example, the rep region represents a repetition or “rep” done by the user during an exercise. For example, if the user is exercising and does a set of ten bicep curls, a rep region represents a single bicep curl of the ten bicep curls. Alternatively, a rep collection region in the raw data is determined 415 based on the set of feature points. A rep collection region includes a plurality of rep regions where each rep region is of a same repetition type, as further described below. Following the bicep curl example, the rep collection region includes ten rep regions where each rep region represents a bicep curl in the set of ten bicep curls.

For purposes of discussion, the following describes determining 415 a rep region but can be extended to determine a rep collection region including the rep region. In one embodiment, if a threshold number of associations have been made between various feature points within the set of feature points, a region including at least one of the various feature points is determined to be a rep region. Following the example before, if the association between two points is a determination of similarity between the points (e.g., the first point is similar to the third point and the second point is similar to the fourth point), then a rep region would include at least the first point but not the third point, at least the second point but not the fourth point, or both the first and second point but not the third point and fourth point. The activity classification server 100 may also determine 415 a rep region to include the first point, the second point, the third point, and the fourth point if the activity classification server 100 knows (e.g., via user input, via pre-classification of the received raw data) that the activity is an activity that includes a complicated motion (e.g., a golf swing including a backward motion, a forward motion, and a second backward motion) that could possibly include repetitive regions (e.g., the backward motion and the second backward motion of the golf swing).

In one embodiment, a rep region in the raw data is determined 415 based on other rep regions in the raw data. For example, if the raw data is an activity region including ten rep regions and eight of the ten rep regions include points with similar patterns or each includes a similar feature point but two of the ten rep regions include points of different patterns, those two rep regions may not be determined as rep regions. Rather, those two rep regions may be determined as regions not of interest. Therefore, if a user is exercising and stops to take a bathroom break, the activity classification server 100 would not identify the bathroom break as a region of interest such as a rep region. Thus, the identified 410 set of feature points in the raw data include at least the eight similar feature points in the eight of the ten rep regions. Other suitable feature points can be points with at least a threshold amplitude (e.g., points associated with highest points in the raw data) or points within a second set of points within an interval of time where the difference in amplitude between the points and the second set of points is greater than a threshold amplitude.

The activity classification server 100 classifies 420 the rep region as a repetition type. In one embodiment, the rep region can be classified as a repetition type based on the threshold number of associations made between various feature points in the rep region. A repetition type can be any movement in an activity such as a sport or an exercise. For example, repetition types in basketball can include dribbling, running, shooting, passing, stepping, and standing. For soccer, repetition types can include kicking, running, walking, standing, and jumping. Further, for golf, repetition types can include a swing, a backward motion, a forward motion, standing, and walking. For certain activities such as golfing, movements can be more complex such as a golf swing. A swing can be a repetition type but the swing can include a backward motion, a forward motion, and a second backward motion. Thus, the rep region can be the swing including the backward motion, forward motion, and second backward motion or each of the separate motions (backward motion, forward motion, and second backward motion) can be rep regions. If the rep region is classified as a swing, the activity classification server 100 can also identify a dominant feature in the rep region such as a highest data point in the rep region which can represent a step in the swing associated with the most movement by the user. Herein, a rep region that is classified as a repetition type of “dribble” can be referred to as a “dribble” rep region.

In one embodiment, the rep region is compared to a reference database of various rep regions previously classified as various repetition types. These can be user-specific reference databases or general reference databases. In alternative embodiments, the feature points in the rep region, the associations between the feature points in the rep region, the rep region, or any combination thereof are compared to the reference database. The reference database can be created using a machine learning classifier using raw data from a plurality of users of the activity-tracking devices 102 and user-provided classifications of the raw data.

Alternatively, the activity classification server 100 prompts the user to perform one or more activities for a threshold amount of time (e.g., 30 seconds) and prompts the user to classify the one or more activities performed such as by selecting an activity from a list and use those classifications as the reference database. In this manner, the user can provide a period of test data that the user has identified as being a particular activity, and the server can use this pre-classified activity to identify that activity in the future. The user can also select to perform one or more activities for a threshold amount of time and classify the one or more activities performed and use those classifications as the reference database. The server 100 can thus collect multiple different types of activity test data from a user that the user classifies as different types of activity, and from this the server then has a basis for identifying those same types of activities in the future. As mentioned above, different users may perform the same type of activity in different ways, so this user classification of activities using test data allows the server to recognize each different user's way of performing the activity. The user's own test data can be associated with the user's profile stored in the user profile store and then used in the future to recognize when that user is performing that activity. For example, user A may go to the gym and do sit-ups, push-ups, bench-presses, and run on the treadmill. The first time that user A performs each of these activities, he can provide a user classification of these activities by selecting a test data collection mode on one of the activity-tracking devices or on a portable computing device 106. He can then perform that activity for a period of time and then indicate via a user interface on one of the devices that the activity was “sit-ups.” This data is sent to the server and stored in association with his user profile. The next time a similar activity pattern is recognized by the server, the server will automatically classify the activity as “sit-ups.”

Since many movements and repetition types are similar, the activity classification server 100 can also calculate a certainty score for various repetition types and classifies the rep region as the repetition type with the highest certainty score. In various embodiments, activity regions and rep regions in the raw data received can be identified or determined using various machine learning algorithms such as pattern recognition algorithms as well as various heuristics to differentiate similar rep regions or to classify a rep region.

Based on the classified rep region as the repetition type, the activity region is associated 425 with an activity type. Following the example from before, when a user uses the plurality of activity-tracking devices 102 while playing basketball, there may be a plurality of rep regions in the activity region of the raw data, such as a “dribble” rep region, five “step” rep regions, and a “shoot” rep region and, based on the combination of those types of repetitions, the activity region is associated with an activity type such as “basketball.” Similarly, when a user uses the plurality of activity-tracking devices 102 while playing basketball, there may be a plurality of rep regions in the activity region of the raw data such as a “kick” rep region, five “run” rep regions, and a “jump” rep region and, based on the combination of those types of repetitions, the activity region is associated with an activity type such as “soccer.”

The activity classification server 100 may also determine that an association of the activity region with an activity type cannot be made based on the classified repetition type. Thus, in response to this determination, the activity classification server 100 can determine an additional rep region in the raw data based on the set of feature points, classify the additional rep region as a repetition type, and associate the activity region with an activity type based on the two classifications of the rep region and the additional rep region. The process described in FIG. 4, specifically steps 415 and 420, can be repeated until the activity region can be associated 425 with an activity type.

### Pre-Classifying Activity Regions, Rep Regions, or Regions in the Raw Data

In some embodiments, the activity classification server 100 can pre-classify the activity region or the rep region in the activity region as a noise region (which includes idle regions), an unintentional activity region, an intentional region, or any other suitable distinguishable classification differing from an activity or being idle. If the activity region or rep region is pre-classified as noise, this region is marked as a region that will not be reported to the user, for example, via a portable computing device 106 associated with the user. As stated previously, the region can be pre-classified as noise (or being an idle region) based on amplitudes of points in the region or lack of repetition of the region in the raw data. If the activity region or rep region is pre-classified as an unintentional activity region, this region is also marked as a region that will not be reported to the user. For example, if the user wiped a whiteboard clean with an eraser, that activity can be identified but it is marked as an unintentional activity region (e.g., based on lack of repetitive regions similar to the region).

If the activity region is pre-classified as an intentional region, this region is marked for further classification and marked to be reported to the user. In one embodiment, after the region has been further classified (e.g., more rep regions are determined and classified), the region and information describing further classification of the region is reported to the user. Alternatively, the activity classification server 100 can report the region to the user and prompt the user to indicate whether further classification should be done on the region, to further classify the region, or any combination thereof.

In other embodiments, if the activity region is pre-classified as an intentional region, the region can be marked to be reported to the user and not to be further classified. For example, if the region is identified as a region that does not require to be further classified (e.g., through lack of additional rep regions in the region or no additional feature points to analyze), the region is marked to not be further classified and, thus, the amount of processing is reduced, saving computation time, money, and additional resources over time.

### Further Determinations for Activity Classification

The activity classification server 100 can also determine activity of a user's upper body, the user's lower body, and any combination thereof while the user is using one or more activity-tracking devices 102. Determining activity of portions of a user's body can help during classification or rep regions as repetition types, classification of activity regions as activity types, of rep regions during pre-classification, or any combination thereof. If the activity classification server 100 determines that no movements are made by the user's lower body, certain repetition types or activity types can be removed during classification and, therefore, reduce computation resources and time. For example, if there are no movements in the user's lower body, then the user is probably not playing a sport that requires movement of the lower body (e.g., running, walking, jogging, jumping, stepping, etc.) nor performing certain exercises (e.g., treadmill, leg extensions, leg lifts, leg curls, etc.). Then, if the activity classification server 100 determines that there are movements in the user's upper body, then certain activity types or repetition types can be associated with a higher certainty score of being the correct classification for the activity regions or rep regions, respectively. Thus, determining whether or not an activity or movement requires movement of the entire body (i.e., upper body and lower body), upper body, or lower body reduces possible matches in activity type and repetition type. For example, the activity classification server 100 can have a hierarchy database including information about whether an activity type or repetition type requires or is associated with movement in the upper body, lower body, or both.

The activity classification server 100 can also determine activity of a user's upper body, the user's lower body, and any combination thereof by temporal information associated with the activity, motion information, scores associated with movement in the user's upper body and/or the lower body, and any combination thereof. Examples of temporal information include a period of time associated with a rep region in the activity region, a period of time associated with the activity region, a start time associated with the activity region, an end time associated with the activity region, a rate associated with a rep region (e.g., how many reps per second), acceleration associated with one or more of the activity-tracking devices 102, and any combination thereof. The period of time associated with a rep region can be a combination of periods of time associated with all similar rep regions in the activity region or received raw data while the user is performing the activity. Similarly, the period of time associated with the activity region can be a combination of periods of time associated with additional activity regions identified in the raw data while the user is performing the activity that are similar to the activity region or previously received activity regions similar to the activity region.

Examples of motion information include an absolute distance traveled by the user while performing a rep region in the activity region, a number of steps taken by the user during a rep region, a number of steps taken by the user during the activity region, one or more muscle targets associated with a rep region, one or more muscle targets associated with the activity region, a number of total repetitive regions associated with a rep region, information describing form of the user while performing the activity, amount of stillness associated with the user while performing the activity, and any combination thereof. The one or more muscle targets associated with the rep region or activity region can be identified based on the activity-tracking devices associated with data streams or points in the raw data including the rep region or activity region. For example, a muscle target can simply be identified as a muscle in the upper body, a muscle in the lower body, or muscles in the upper and lower body. The information describing form of the user while performing the activity can be based on location information of the one or more activity-tracking devices 102 used by the user while performing the activity. For example, the information can identify how far apart the user's feet are relative to the user's height, how far apart the user's hands are relative to the user's height, and any other suitable relative information of the activity-tracking devices 102.

Examples of scores associated with movement in the user's upper and/or lower body include a number of rep regions that have a certainty score exceeding a threshold score, a number of rep regions that have a certainty score not exceeding a threshold score, a consistency score of a rep region in the activity region, an intensity score associated with the activity region, an intensity score associated with the rep region, an absolute distance traveled by the user while performing the activity, and any combination thereof. The consistency score of a rep region in the activity region can be a score based on a number of rep regions in the activity region and a number of rep regions in a previously received activity region that is similar to the activity region. For example, if the user previously did five reps of a set of ten bicep curls for a month and then did three reps of a set of ten bicep curls or did five reps of a set of three bicep curls one day after the month, then the consistency score of the user would decrease. The intensity score can be based on a number of reps, a number of sets in a rep, and any combination thereof. For example, the intensity score can be proportional to the number of reps and the number of sets in a rep.

### Activity Regions, Rep Regions, and Feature Points in Raw Data

FIG. 5 is an example of raw data 500 including an activity region 550, an idle region 560, and a rep region 570 and feature points 580 in the raw data 500, in accordance with an embodiment. The raw data 500 shown in FIG. 5 includes two data streams 505A and 505B associated with each of two activity-tracking devices 102A and 102B, respectively. For example, data stream 505A is associated with an activity-tracking device 102B worn on a user's lower body and data stream 505B is associated with an activity-tracking device 102A worn on the user's upper body.

Data stream 505A includes an activity region 550A with at least four rep regions 570A, 570B, 570C, and 570D. Each rep region 570A-D includes one or more feature points 580 and, as an example, rep region 570A includes feature points 580A, 580B, 580C, 580D, 580E, and 580F where a feature point is associated with a time and an amplitude and one or more of the feature points are associated with at least a feature point in another rep region 570. For example, feature point 580A is associated with at least feature points 580G, 580H, and 580J.

Data stream 510B includes two activity regions 550B and 550C as well as an idle region 560. Activity region 550B includes two rep regions 570E and 570F and activity region 550C includes activity region 570G. Each rep region 570E-G also includes one or more feature points 580 and, as an example, rep region 570E includes feature points 580K, 580L, 580M, 580N, 580P, and 580Q. Each one of the feature points in 570E is associated with at least a feature point in another rep region 570. For example, feature point 580K is associated with at least feature points 580R and 580S. The idle region 560 can also be associated with feature points such as feature points 580T and 580U.

### Variation in Feature Points Included in Raw Data

FIG. 6 is an example of feature points 680 in raw data 600 indicating a repetition is associated with muscle fatigue or higher resistance, in accordance with an embodiment. The raw data 600 can include a plurality of data streams 605 but the example shown in FIG. 6 includes one data stream 605. The data stream can include one or more activity regions 605 and one or more idle regions but the example shown in FIG. 6 includes one activity region 605 including a plurality of similar rep regions 670A, 670B, 670C and 670D. A rep region 670 can include one or more feature points and, as an example, rep region 670A includes feature points 680A, 680B, 680C, 680D, 680E, and 680F where a feature point is associated with a time and an amplitude and one or more of the feature points are associated with at least a feature point in another rep region 670. For example, feature point 680A is associated with at least feature points 680G, 680H, and 680J.

The activity classification server 100 can determine that the repetition of the rep regions 670A-D are associated with either muscle fatigue or higher resistance based on comparisons of feature points 680 in the rep regions 670 associated with each other. For example, amplitude of the feature points 680 in the rep regions 670 associated with each other can be compared. For example, feature points 680A, 680G, 680H, and 680J are feature points 680 in separate rep regions 670 that are associated with each other. However, the amplitude of the feature points 680A, 680G, 680H, and 680J decrease over time, as shown in FIG. 6. Thus, the activity classification server 100 can determine that the decrease in amplitude can be associated with muscle fatigue or higher resistance.

In alternative embodiments, duration of each rep region 670 can be used to determine muscle fatigue or higher resistance. As shown in FIG. 6, each rep region 670 is associated with an interval of time that exceeds an interval of time associated with a previous rep region 670. For example, rep region 670B is longer than rep region 670A, rep region 670C is longer than rep region 670B, and rep region 670D is longer than rep region 670C. Therefore, the activity classification server 100 can also determine that the increase in the interval of time with each subsequent rep region 670 can be associated with muscle fatigue or higher resistance.

### Summary

The foregoing description of the embodiments has been presented for the purpose of illustration; it is not intended to be exhaustive or to limit the patent rights to the precise forms disclosed. Persons skilled in the relevant art can appreciate that many modifications and variations are possible in light of the above disclosure.

Some portions of this description describe the embodiments in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

Any of the steps, operations, or processes described herein may be performed or implemented with one or more hardware or software modules, alone or in combination with other devices. In one embodiment, a software module is implemented with a computer program product comprising a computer-readable medium containing computer program code, which can be executed by a computer processor for performing any or all of the steps, operations, or processes described.

Embodiments may also relate to an apparatus for performing the operations herein. This system includes apparatuses that may be specially constructed for the required purposes (e.g., specially designed activity tracking devices for tracking certain activities), and it may include one or more general-purpose computing devices selectively activated or reconfigured by a computer program stored in the computer. Such a computer program may be stored in a non-transitory, tangible computer readable storage medium, or any type of media suitable for storing electronic instructions, which may be coupled to a computer system bus. Furthermore, any computing systems referred to in the specification may include a single processor or may be architectures employing multiple processor designs for increased computing capability.

Embodiments may also relate to a product that is produced by a computing process described herein. Such a product may comprise information resulting from a computing process, where the information is stored on a non-transitory, tangible computer readable storage medium and may include any embodiment of a computer program product or other data combination described herein.

Finally, the language used in the specification has been principally selected for readability and instructional purposes, and it may not have been selected to delineate or circumscribe the inventive subject matter. It is therefore intended that the scope of the patent rights be limited not by this detailed description, but rather by any claims that issue on an application based hereon. Accordingly, the disclosure of the embodiments is intended to be illustrative, but not limiting, of the scope of the patent rights, which is set forth in the following claims.

What is claimed is:

1. A method comprising:
receiving, by an activity classification server, raw data from a plurality of activity-tracking devices worn by a user while performing an activity, the raw data comprising at least one activity region with one or more points, each point associated with a time stamp and amplitude;
identifying an activity region in the raw data, the activity region comprising at least a threshold number of points associated with amplitudes that exceed a first threshold amplitude within an interval of time;
identifying a set of feature points in the activity region in the raw data, a feature point in the set of feature points associated with an amplitude that exceeds a second threshold amplitude, at least one feature point in the set of feature points similar in amplitude to a second feature point in the set of feature points;
determining a repetition (“rep”) region in the activity region in the raw data based on the set of feature points, the rep region including the at least one feature point and not including the second feature point;
classifying the rep region as a repetition type, a repetition type being a movement associated with an activity.

2. The method of claim 1, further comprising:
performing a preliminary analysis on the received raw data to identify repetitive regions, a repetitive region associated with at least a threshold interval of time and comprising a threshold number of points repeated in a second threshold interval of time in the raw data.

3. The method of claim 2, further comprising:
presenting the user with information based on the preliminary analysis, the information describing change in muscle fatigue over the repetitive regions, change in resistance between repetitive regions, or any combination thereof.

4. The method of claim 2, further comprising:
presenting the user with information based on the preliminary analysis, the information describing suggestions based on changes between repetitive regions, or any combination thereof.

5. The method of claim 1, wherein an identified feature point associated with a time stamp is further identified based on a second feature point associated with a second time stamp that is an interval from the time stamp.

6. The method of claim 1, wherein determining a rep region in the activity region in the raw data based on the set of feature points further comprises:
determining a number of associations between various feature points in the set of feature points; and
responsive to the number of associations exceeding a threshold number of associations, determining a region in the activity region in the raw data including at least a feature point in the various feature points as the rep region.

7. The method of claim 6, wherein the rep region is classified as a repetition type based on the number of associations.

8. The method of claim 1, wherein classifying the rep region as a repetition type comprises:
comparing feature points in the rep region with a reference database, the reference database comprising associations between rep regions previously performed by a plurality of users of the activity classification server and determined classifications of the rep regions.

9. The method of claim 1, further comprising:
prompting the user wearing the plurality of activity-tracking devices to record test data for an activity about to be performed by the user for a predetermined interval of time;
receiving preliminary raw test data tracking the prompted activity;
prompting the user to classify the prompted activity; and
storing the classification and the preliminary raw data in a reference database.

10. The method of claim 9, wherein classifying the rep region as a repetition type comprises:
comparing feature points in the rep region with the reference database.

11. The method of claim 1, further comprising:
associating the activity region in the raw data with an activity type based on the classified rep region as the repetition type.

12. The method of claim 1, further comprising:
determining that an association of the activity region cannot be made based on the classified rep region;
determining an additional rep region in the activity region in the raw data based on the set of feature points;
classifying the additional rep region as a second repetition type; and
associating the activity region in the raw data with an activity type based on the classified rep region as the repetition type and the classified additional rep region as the second repetition type.

13. A method comprising:
receiving, by an activity classification server, raw data from a plurality of activity-tracking devices worn by a user while performing an activity, wherein the raw data comprises one or more points, each point associated with a time stamp and an amplitude exceeding a first threshold amplitude;
identifying a set of feature points in the raw data, a feature point in the set of feature points associated with an amplitude that exceeds a second threshold amplitude, at least one feature point in the set of feature points similar in amplitude to a second feature point in the set of feature points;
determining a repetition (“rep”) region in the raw data based on the set of feature points, the rep region including the at least one feature point and not including the second feature point;
classifying the rep region as a repetition type, a repetition type being a movement associated with an activity; and
associating the rep region with an activity type based on the classified rep region as the repetition type.

14. The method of claim 13, wherein an identified feature point associated with a time stamp is further identified based on a second feature point associated with a second time stamp that is an interval from the time stamp.

15. The method of claim 13, wherein determining a rep region in the raw data based on the set of feature points further comprises:
determining a number of associations between various feature points in the set of feature points; and
responsive to the number of associations exceeding a threshold number of associations, determining a region in the raw data including at least a feature point in the various feature points as the rep region.

16. The method of claim 15, wherein the rep region is classified as a repetition type based on the number of associations.

17. The method of claim 13, wherein classifying the rep region as a repetition type comprises:
comparing feature points in the rep region with a reference database, the reference database comprising associations between rep regions previously performed by a plurality of users of the activity classification server and determined classifications of the rep regions.

18. The method of claim 13, further comprising:
prompting the user wearing the plurality of activity-tracking devices to record test data for a repetition of an activity type about to be performed by the user for a predetermined interval of time;
receiving preliminary raw test data tracking the prompted repetition;
prompting the user to classify the prompted repetition as a repetition type; and
storing the classification and the preliminary raw data in a reference database.

19. The method of claim 18, wherein classifying the rep region as a repetition type comprises:
comparing feature points in the rep region with the reference database.

20. A non-transitory computer-readable storage medium having instructions encoded thereon that, when executed by a processor, cause the processor to:
receive raw data from a plurality of activity-tracking devices worn by a user while performing an activity, wherein the raw data comprises one or more points, each point associated with a time stamp and an amplitude exceeding a first threshold amplitude;
identify a set of feature points in the raw data, a feature point a point in the set of feature points associated with an amplitude that exceeds a second threshold amplitude, at least one feature point in the set of feature points similar in amplitude with a second feature point in the set of feature points;
determine a repetition (“rep”) region in the raw data based on the set of feature points, the rep region including the at least one feature point and not including the second feature point;
classify the rep region as a repetition type, a repetition type being a movement associated with an activity; and
associate the rep region with an activity type based on the classified rep region as the repetition type.
